(19) United States
(12) Patent Application Publication
(10) Pub. No.: US 2004/0101554 A1
(43) Pub. Date: May 27, 2004

(54) Nutritional formulations

(75) Inventors: Mitchell I. Kirschner, St. Louis, MO (US); R. Saul Levinson, Chesterfield, MO (US); Marc S. Hermelin, Glendale, MO (US)

Correspondence Address: Richard H. Shear, Blackwell Sanders Peper Martin, LLP, 24th Floor, 720 Olive Street, St. Louis, MO, 63101, US

(73) Assignee: DrugTech Corporation, Wilmington, DE (US)

(21) Appl. No.: 10/714,156

(22) Filed: Nov. 14, 2003

Related U.S. Application Data
(63) Continuation of application No. 09/972,664, filed on Oct. 9, 2001, now abandoned.
Application No. 09/972,664 is a continuation-in-part of application No. 09/320,559, filed on May 27, 1999, now abandoned.

(51) Int. Cl. A61K 9/64; A61K 31/22; A61K 31/525

(52) U.S. Cl. 424/456; 514/549; 514/251

(57) ABSTRACT

This invention relates to novel dosage formulations for nutritional compositions comprising fatty acids derived from both plant and animal sources and methods for minimizing unpleasant taste, regurgitation, gastroesophageal reflux, dyspepsia, nausea, or difficulty in swallowing or ingesting nutritional agents. The nutritional compositions are intended for use by pregnant or lactating women.

### CROSS REFERENCE TO RELATED APPLICATIONS

This patent application is a continuation application of U.S. patent application Ser. No. 09/972,664 filed Oct. 9, 2001, which is hereby incorporated by reference and is currently pending, which claims priority to U.S. patent application Ser. No. 09/320,559 filed May 27, 1999, now abandoned.

### BACKGROUND OF THE INVENTION

1. Field of the Invention

This invention is directed to novel soft gelatin nutritional supplements, particularly soft gelatin nutritional supplements for pregnant women comprising fatty acids, methods of using said supplements to reduce the unpleasant taste, regurgitation, gastroesophageal reflux, dyspepsia, and nausea associated with the administration of traditional prenatal nutritional supplements, and processes for manufacturing said supplements.

2. Description of the Related Art

Gastrointestinal motility problems are common in women at all stages of pregnancy. Approximately 45% to 85% of women report experiencing digestive disturbances during pregnancy. Olans, et al., “Gastroesophageal reflux in pregnancy”, Gastrointest Endosc Clin N Am 4(4):699-712 (1994). Typical symptoms experienced by pregnant women include belching, heartburn, gastroesophageal reflux; dyspepsia, regurgitation, increased sensitivity to unpleasant odors and/or tastes, nausea and vomiting. The Merck Manual, 1850-1866 (16th Ed. 1992). These symptoms are thought to be brought about, in part, by the physiological changes which occur in the female body during pregnancy.

As pregnancy progresses, gastrointestinal motility decreases due to elevated progesterone levels which cause the smooth muscles associated with the digestive tract to relax. Id. The delay in gastric emptying time and relaxation of the sphincter located at the junction of the esophagus and stomach can cause a reflux of gastric fluids into the esophagus, e.g. gastroesophogeal reflux. Id. The relaxation of the diaphragmatic hiatus can exacerbate this condition. Id.

The caustic nature of the refluxate and the inability to clear the refluxate from the esophagus can cause heartburn or heartburn-like symptoms. Id. In some instances, the heartburn symptoms will be accompanied by regurgitation of the gastric contents into the mouth. The Merck Manual, 1850-1866 (16th Ed. 1992).

The condition of gastroesophageal reflux may be self-perpetuating if not managed and/or treated. Because of the caustic properties of the gastric contents, repeated esophageal exposure to these substances can lead to a permanent incompetence of the esophageal sphincter. Id. Furthermore, in more serious cases, esophagitis, peptic esophageal stricture, esophageal ulcer, and Battert's metaplasia can result in a case of complicated gastroesophageal reflux. Id. Therefore, management and therapy of the condition are of the utmost priority.

The gastrointestinal disturbances associated with pregnancy are normally mild in degree and viewed as a natural part of the pregnancy experience. However, these facts do not lessen the discomfort experienced by pregnant women or the seriousness of the potential complications of the condition. Furthermore, as with any course of medical treatment in pregnant women, a primary concern is the potential teratogenicity of the proposed drug therapy. Many gastrointestinal medications are either known teratogens or have not been adequately studied with regards to their effect upon pregnant humans.

It has been noted that medications used in the treatment of gastroesophageal reflux are not routinely or vigorously tested in randomized, controlled trials in pregnant women because of ethical and medico-legal concerns. Broussard, et al. “Treating gastro-esophageal reflux disease during pregnancy and lactation: what are the safest therapy options,” Drug Saf, 19(4):325-37 (1998). For example, the cholinergic antagonist Cystospaz®, available from PolyMedica Pharmaceuticals (U.S.A.), Inc., which is of the class of drugs which can be prescribed for gastroesophageal reflux due to their positive effect upon esophageal sphincter pressure, is not recommended for use in pregnant women, because animal reproductive studies have not been conducted. Furthermore, “it is not known whether CYSTOSPAZ® Tablets or CYSTOSPAZ-M® Capsules, can cause fetal harm when administered to a pregnant woman.” Physicians' Desk Reference, 2526-7 (53d Ed. 1999).

Other cholinergic antagonists are provided with similar precautions. Donnatal®, available from A.H. Robins Company, is not recommended for administration to pregnant women due to the lack of adequate animal reproduction studies, and also because the effect of the drug on the fetus is not known. Id. at 2636. Kutrase®, available from Schwarz Pharma, Inc., Levsin®, also available from Schwarz Pharma, Inc. and Robaxisal®, available from A.H. Robins Company, all carry similar precautions regarding prescription to pregnant and/or lactating women. Id at 2907; See also, Id. at 2910; See also, Id. at 2646.

As a result, most physicians initially begin managing gastrointestinal disturbances in pregnant women with aggressive lifestyle modification and dietary changes rather than drug therapy. Katz, et al., “Gastroesophageal reflux disease during pregnancy,” Gastroenterol Clin North Am, 27(1):153-67 (1998). While this course of therapy is primarily due to the concern of exposing the fetus to teratogenic substances via drug therapy, it has been discovered that lifestyle and dietary management are often extremely effective in precipitating relief. Katz, et al. “Gastroesophageal reflux disease during pregnancy,” Gastroenterol Clin North Am 27(1): 153-67 (1998).

Dietary management consists of isolating those foods or classes of foods which bring about the symptoms of gastroesophageal reflux. The Merck Manual, 749 (16th Ed. 1992). Typically, the common foods which aggravate the condition are fried or fatty foods, caffeinated beverages or foods, for example coffee and chocolate, and spicy foods. It is thought that these foods stimulate acid production and/or reduce lower esophageal sphincter competence. Id.; See also, Nebel, et al., “Symptomatic gastroesophageal reflux: incidence and precipitating factors,” Am J Dig Dis, 21(11) :953-6 (1976).

Furthermore, it has been discovered that gastrointestinal relief can be brought about by directing the pregnant woman to eat small portions at frequent intervals and to increase the amount of carbohydrates while simultaneously decreasing her fat intake. Morton, “Treating nausea and vomiting in pregnancy,” Am Fam Physician, 48(7):1279-84 (1993). Other general recommendations include instituting a bland diet, avoiding bothersome food odors and omitting. prenatal vitamins from the dietary regimen. Id.

The omission of prenatal vitamins is a problematic recommendation for the pregnant woman. While it is acknowledged that vitamin supplements can cause uncomfortable gastrointestinal effects, i.e., gagging, regurgitation, gastroesophageal reflux, dyspepsia, and/or nausea, and can be unpleasant to take due to taste, smell, size and/or the texture of the tablet, it is also a well established fact that pregnant women have heightened nutritional requirements. A mother's body provides the environment in which development of the embryo and fetus occur. See Understanding Nutrition, 479-480 (Whitney and Rolfes Eds. 6th Ed., 1993). Accordingly, the mother's nutritional status during pregnancy directly impacts the development of the fetus and embryo and is therefore implicated with regard to the occurrence of birth defects. See Id.

In particular, during the first 20-25 days of pregnancy, the placenta is not yet formed and fetal circulation is not yet established. Therefore, during this period the fetus is nourished via digested maternal uterine cells and the diffusion of blood exudates. See Schorah “Importance of Adequate Folate Nutrition in Embryonic and Early Fetal Development,” Vitamins and Minerals in Pregnancy and Lactation, 167-176 (Berger, Ed., Vol. 16, 1988). It is believed that a good nutrient supply during the first 20-25 days of pregnancy is necessary to provide optimal concentrations of essential micronutrients to the endometrium. See Id.

Furthermore, increased occurrences of birth defects have been linked to inadequate maternal nutrition. Cases of infants born with a neural tube defect, i.e., spina bifida or anacephaly, have been documented in women with various nutritional deficiencies, primarily low blood folic acid and vitamin C concentrations. Smithells, “Vitamin deficiencies and neural tube defects,” Arch Dis Child, 51:944-50 (1976).

The presence of fatty acids in nutritional supplements is significant for various reasons, as described below. First, the body derives most of its energy from triglycerides, a molecule of glycerol with three fatty acids attached. The stored fatty acids support most of life's activities when individual's are between meals or must go without food. While the body can make many fatty acids, it cannot make linoleic acid or linolenic acid. These two fatty acids are indispensable to body functions and therefore must be supplied through food.

Secondly, essential fatty acids are important for the developing brain, immunological system and cardiovascular system, and have some role to play in every organ of the body of the fetus. Linoleic, acid is the most important member of the omega-6 family of fatty acids. The body uses linoleic acid to synthesize an important 20-carbon fatty acid, arachidonic acid, which helps maintain the structural integrity of cell membranes.

Linolenic acid is the most important member of the omega-3 family of fatty acids. The body requires this fatty acid to make eicosapentaenoic acid (EPA) and docosahexaenoic acid (DHA). Many body tissue require EPA and DHA. DHA is especially important in the retina and in the cerebral cortex of the brain. Half of the DHA in a fetus' body accumulates in the brain before birth, and half after birth, an indication of the important of fatty acids to the fetus during pregnancy and then to the young infant during lactation.

The importance of the nutritional status of pregnant women is evident in the number of prenatal vitamins currently available. The Physicians' Desk Reference describes various vitamin and mineral supplements for use by pregnant women. For example, Nestabs® CBF prenatal formula, available from The Fielding Company, contains 4,000 I.U. of vitamin A, 400 I.U. of vitamin D, 30 I.U. of vitamin E, 120 mg Of vitamin C, 1 mg of folic acid, 3 mg of thiamine, 3 mg of riboflavin, 20 mg of niacinamide, 3 mg of pyridoxine, 8 mcg of vitamin B₁₂, 20 mg of calcium, 100 mcg of iodine, 15 mg of zinc, and 50 mg of iron per dose. NESTABS® CBF are “expressly formulated for use during pregnancy and lactation” and are available only in tablet form. See Physicians' Desk Reference, 1011 (53d Ed., 1999).

Materna®, prenatal vitamin and mineral formula, available from Lederle Laboratories, contains 5,000 I.U. of vitamin A, 400 I.U. of vitamin D, 30 I.U. of vitamin E, 120 mg of vitamin C, 1 mg of folic acid, 3 mg of vitamin B₁, 3.4 mg of vitamin B₂, 10 mg of vitamin B₆, 20 mg niacinamide, 12 mcg of vitamin B₁₂, 30 mcg of biotin, 10 mg of pantothenic acid, 200 mg of calcium, 150 mcg of iodine, 27 mg of iron, 25 mg of magnesium, 2 mg of copper, 25 mg of zinc, 25 mg of chromium, 25 mg of molybdenum, 5 mg of manganese, and 20 mcg of selenium per dose. Materna® is designed “to provide vitamin and mineral supplementation prior to conception, throughout pregnancy and during the postnatal period for both lactating and nonlactating mothers” and is available in tablet form only. See Id. at 1522-3.

Enfamil® Natalins® RX multivitamin and multimineral supplement, available from Mead Johnson Nutritionals, Mead Johnson & Company, provides 4000 I.U. of vitamin A, 80 mg of vitamin C, 400 I.U. of vitamin D, 15 I.U. of vitamin E, 1.5 mg of thiamin, 1.6 mg of riboflavin, 17 mg niacin, 4 mg of vitamin B₆, 1 mg of folic acid, 2.5 mcg of vitamin B,₂, 30 mcg of biotin, 7 mg of pantothentic acid, 200 mg of calcium, 54 mg of iron, 25 mg of zinc, and 3 mg of copper per dose. Enfamil® Natalins® RX are formulated “to supplement the diet during pregnancy of lactation” and are available only in tablet form. See Id. at 1692.

Prenate® Ultra™ prenatal vitamins, available from Sanofi Pharmaceuticals, Inc., contain 90 mg of elemental iron, 150 mcg of iodine, 200 mg of calcium, 2 mg of copper, 25 mg of zinc, 1 mg of folic acid, 2700 I.U. of vitamin A, 400 I.U. of vitamin D₃, 30 I.U. of vitamin E, 120 mg of vitamin C, 3 mg of vitamin B₁, 304 mg of vitamin B₂, 20 mg of vitamin B₆, 12 mcg of vitamin B₁₂, 20 mg of niacinamide, and 50 mg of docusate sodium per dose. Prenate® Ultra™ is “indicated for use in improving the nutritional status of women throughout pregnancy and in the postnatal period for both lactating and nonlactating mothers” and is only available in tablet form. See Id. at 2802.

Niferex®-PN formula, available from Schwarz Pharmaca, Inc., contains 60 mg of iron, 1 mg of folic acid, 50 mg of vitamin C, 3 mcg of vitamin B₁₂, 4,000 1.0. of vitamin A, 400 1.0. of vitamin D, 2.43 mg of vitamin B₁, 3 mg of vitamin B₂, 1.64 mg of vitamin B₆, 10 mg of niacinamide, 125 mg of calcium, and 18 mg of zinc per dose. Niferex®-PN is “indicated for prevention and/or treatment of dietary vitamin and mineral deficiencies associated with pregnancy and lactation” and is only available in tablet form. See Physicians' Desk Reference, (53d Ed., 1999) 2916-7.

Niferex®-PN Forte formula, also available from Schwarz Pharmaca, Inc., contains 60 mg of iron, 1 mg of folic acid, 50 mg of vitamin C, 3 mcg of vitamin B₁₂, 5,000 1.0. of vitamin A, 400 1.0. of vitamin D, 30 1.0. of vitamin E, 80 mg of vitamin C, 1 mg of folic acid, 3 mg of vitamin B₁, 3.4 mg of vitamin B₂, 4 mg of vitamin B₆, 20 mg of niacinamide, 12 mcg of vitamin B₁₂, 250 mg of calcium, 200 mcg of iodine, 10 mg of magnesium, 2 mg of copper, and 25 mg of zinc per dose. Niferex®-PN is “indicated for prevention and/or treatment of dietary vitamin and mineral deficiencies associated with pregnancy and lactation” and is only available in tablet form. See Id. at 2917-8.

Advanced Formula Zenate® prenatal multivitamin/mineral supplement, available from Solvay Pharmaceuticals, Inc., contains 3,000 1.0. of vitamin A, 400 1.0. of vitamin D, 10 I.U. of vitamin E, 70 mg of vitamin C, 1 mg of folic acid, 1.5 mg of vitamin B₁, 1.6 mg of vitamin B₂, 17 mg of niacin, 2.2 mg of vitamin B₆, 2.2 of vitamin B₁₂, 200 mg of calcium, 175 mcg of iodine, 65 mg of iron, 100 mg of magnesium, and 15 mg of zinc per dose. Advanced Formula Zenate® is “a dietary adjunct in nutritional stress associated with periconception, pregnancy and lactation” and is only available in tablet form. See Id. at 3128.

Precare® prenatal multi-vitamin/mineral formula, available from UCB Pharma, Inc., contains 50 mg of vitamin C, 250 mg of calcium, 40 mg of iron, 6 mcg of vitamin D, 3.5 mg of vitamin E, 2 mg of vitamin B₆, 1 mg of folic acid, 50 mg of magnesium, 15 mg of zinc and 2 mg of copper per dose. Precare® “is indicated to provide vitamin and mineral supplementation throughout pregnancy and during the postnatal period-for both lactating and nonlactating mothers” and is available only in caplet form. See Id. at 3163.

Natafort@ prenatal multivitamin, available from Warner Chilcott Laboratories, contains 1,000 1.0. of vitamin A, 400 1.0. of vitamin D₃, 11 IU.. of vitamin E, 120 mg of vitamin C, 1 mg of folic acid, 2 mg of thiamine mononitrate, 3 mg of riboflavin, 20 mg of niacinamide, 10 mg of vitamin B₆, 12 mcg of vitamin B₁₂, and 60 mg of iron per dose. Natafort® is designed “to provide vitamin and mineral supplementation throughout pregnancy and during the postnatal period, for both the lactating and non-lactating mother” and is only available in tablet form. See Id. at 3212.

Soft gelatin dosage forms are flexible, one-piece, hermetically sealed soft shells, comprised of gelatin, a plasticizer, and a small quantity of water and which contains a fill, of one or more active ingredients in combination to form a liquid, suspension or a semi-solid center. Soft gelatin technology has been previously described in various references. For example, Yu et al., U.S. Pat. No. 5,071,643, disclose a solvent system for enhancing the solubility of acidic, basic, or amnphoteric pharmaceutical agents to produce a highly concentrated solution suitable for soft gelatin filling or two piece encapsulation. The solvent system comprises polyethylene glycol containing 0.2-1.0 mole equivalent pharmaceutical agent and 1-20% water. Glycerin or polyvinylpyrrolidone may be added to further enhance the solubility of certain drugs. The solvent system is capable of enhancing the solubility of pharmaceutical agents 40-400%.

Stone, U.S. Pat. No. 5,827,535, discloses a soft gelatin bearing an impressed graphic representation, such as a letter, name, logo, pictorial representation and the like and a method for making such a soft gelatin.

Ratko et al., U.S. Pat. Nos. 5,422,160 and 5,246,635, disclose a soft gelatin having a texture on at least a portion of its surface and a process and apparatus for the manufacture of such a soft gelatin.

Steele et al., U.S. Pat. No. 5,200,191, disclose a soft gelatin manufacturing process comprising subjecting encapsulated soft gelatins to a stress relieving step, wherein the soft gelatins are placed in a drying tunnel and exposed to heightened temperature and humidity conditions.

Coapman et al., U.S. Pat. No. 5,141,961, disclose a process for solubilizing difficultly soluble pharmaceutical actives in a mixture of polyethylene glycol and polyvinylpyrolidone in the absence of external heat or water.

Cimiluca, U.S. Pat. No. 5,641,512, discloses a soft gelatin capsule composition comprising an analgesic in a soft shell containing a xanthine derivative, such as caffeine.

Yu et al., U.S. Pat. No. 5,360,615, disclose a solvent system for enhancing the solubility of acidic, basic, or amphoteric pharmaceutical agent to produce a highly concentrated solution suitable for soft gelatin filling or two piece encapsulation. The solvent system comprises polyethylene glycol containing 0.2-1.0 mole equivalents of an ionizing agent per mole equivalent pharmaceutical agent and 1-20% water.

The compositions and methods discussed above are deficient in various aspects. Primarily, the compositions are not specifically formulated for administration of fatty acids in soft gelatin dosage form. Even the above discussed references, which recognize the need for an easier to swallow form of prenatal vitamin, are limited to coated tablet or caplet forms and are not optimal for minimizing unpleasant taste and/or smell, regurgitation, gastroesophageal reflux, dyspepsia, and/or nausea and maximizing ease of swallowing or ingestion. Furthermore, the soft gelatin formulations which are discussed do not offer any guidance with regard to formulating specific nutritional compositions containing fatty acids for the prenatal patient. Thus, these references are inadequate with regard to improving oral vitamin and mineral supplement administration in the prenatal patient. Finally, previously disclosed compositions do not provide guidance with regard to optimal means of achieving a biologically-active soft gelatin dosage form of prenatal vitamin.

Therefore, there remains a need for a soft gelatin prenatal vitamin and mineral supplement which delivers fatty acids and has a minimal negative effect upon the gastrointestinal tract of the patient, as well as supports the general health of the patient. Moreover, there is a particular need for soft gelatin formulations which promote the good health of the expectant mother and are pleasant to ingest, and thus will provide a higher degree of patient compliance while simultaneously minimizing the cost to the patient.

It is also particularly desirable to have available formulations for addressing the nutritional needs of pregnant women which are designed to have a minimized impact upon the gastrointestinal system, specifically by providing a formulation which delivers fatty acids over an extended period of time. Because of the sensitive nature of this system during pregnancy and the desire to reduce or avoid medication during pregnancy, such soft gelatin formulations are advantageous in that they do not provoke gastrointestinal disturbances. Thus, there is a general overall need for a fundamentally new, safe and effective approach to addressing the physiological needs of pregnant women required to or desirous of partaking in a prenatal vitamin and mineral regimen but are unable to do so because of gastrointestinal system sensitivity.

### SUMMARY OF THE INVENTION

The present inventive subject matter overcomes the shortcomings of currently available prenatal supplements by providing fatty acid nutritional compounds in soft gelatin form. The present inventive subject matter also satisfies specific vitamin, mineral and/or nutrient requirements, the absence of which have been found to cause birth defects, as well as to provide for general health during pregnancy. The formulations of the inventive subject matter have been found to optimize the health benefits to pregnant women while minimizing unpleasant taste, regurgitation, gastroesophageal reflux, dyspepsia, nausea, or difficulty swallowing or ingesting nutritional agents. The compositions of the inventive subject matter include certain nutritional components in dosage levels found to optimize fetal development.

The supplements of the present inventive subject matter are comprised of various nutritional compounds dissolved in suspension. By providing nutritional compositions in suspension, rather than in solid form, the number of digestive steps performed by the body is reduced, and the nutritional compounds are therefore more readily available for use by the body. Moreover, the stresses to the gastrointestinal tract are decreased.

Further mineral compounds such as calcium are predispersed in suspension, thereby obviating the need for the digestive system to dissolve the supplement as would be the case with other dosage forms. Accordingly, the actives of the supplement are more quickly available to the body when in soft gelatin form.

Thus, the inventive subject matter provides a soft gelatin nutritional supplement for administration to a pregnant or lactating woman for the purpose of minimizing unpleasant taste, regurgitation, gastroesophageal reflux, dyspepsia, nausea, or difficulty in swallowing or ingesting nutritional agents, which comprises: an omega-3 fatty acid precursor, derivative thereof, or mixtures thereof; an omega-6 fatty acid, a derivative thereof, or mixtures thereof; an omega-6 fatty acid precursor or derivatives or mixtures thereof; wherein the weight ratio of said precursor of omega-3 fatty acid and said precursor of omega-6 fatty acid to said omega-6 fatty acid is about 1:2.5 to 3.0; and a soft gelatin shell.

The inventive subject matter further provides for a soft gelatin nutritional supplement for administration to a pregnant or lactating woman for the purpose of minimizing unpleasant taste, regurgitation, gastroesophageal reflux, dyspepsia, nausea, or difficulty in swallowing or ingesting nutritional agents, which comprises: an omega-3 fatty acid precursor, a derivative thereof, or mixtures thereof; an omega-6 fatty acid, a derivative thereof, or mixtures thereof; an omega-6 fatty acid precursor or derivatives thereof or mixtures thereof, a calcium compound, a derivative thereof or mixtures thereof in an amount ranging from about 10 mg to about 2,000 mg; wherein the weight ratio of said precursor of omega-3 fatty acid and said precursor of omega-6 fatty acid to said omega-6 fatty acid is about 1:2.5 to 3.0; and a soft gelatin shell.

The inventive subject matter also provides for a method for reducing unpleasant taste, regurgitation, gastroesophageal reflux, dyspepsia, or nausea associated with the administration of nutritional supplements, which comprises: orally administering to a pregnant or lactating woman a soft gelatin capsule, wherein said soft gelatin capsule comprises: an omega-3 fatty acid precursor, a derivative thereof, or mixtures thereof, an omega-6 fatty acid, a derivative thereof, or mixtures thereof; an omega-6 fatty acid precursor or a derivative or mixtures thereof; wherein the weight ratio of said precursor of omega-3 fatty acid and said precursor of omega-6 fatty acid to said omega-6 fatty acid is about 1:2.5 to 3.0.

Thus the inventive subject matter provides for the administration of fatty acids and the minimization of the unpleasantness normally associated with taking nutritional supplements during pregnancy.

### DETAILED DESCRIPTION OF THE INVENTION

As used herein, “soft gelatin” refers to a one-piece, hermetically sealed soft gelatin shell containing a fill, in particular a liquid, a suspension or a semi-solid.

“Unpleasant taste” refers to the bothersome taste normally associated with oral dosage forms containing nutritional compounds.

“Difficulty in swallowing or ingestion” refers to the hindered ability to orally consume nutritional compounds primarily due to the supplement's unpleasant taste and/or smell, gastrointestinal sensitivity or some other incompatibility between the patient's physiology and the physical properties of the nutritional compounds, without limitation.

“Biologically active substance” refers to any substance or substances comprising a drug, active therapeutic substance, metabolite, medicament, vitamin, or mineral, any substance used for treatment, prevention, diagnosis, cure or mitigation of disease or illness, any substance which affects anatomical structure or physiological function, or any substance which alters the impact of external influences on an animal, or metabolite thereof, and as used herein, encompasses the terms “active substance”, “therapeutic substance”, “lagent”, “lactive agent”, “active therapeutic agent”, “drug”, “medication”, “medicine”, “medicant”, and other such similar terms.

“Biologically-active core composition” refers to a liquid, suspension or semi-solid composition which is contained within the soft gelatin coating and is comprised of nutritional compound suspended in an edible oil or polymer and which further may be used for treatment, prevention, diagnosis, cure or mitigation of disease or illness, to effect anatomical structure or physiological function, or alter the impact of external influences upon the body.

“Biologically-acceptable” refers to being safe for human consumption.

“Nutritional compound” refers to any compound which provides nourishment to cells of the body, including without limitation: any vitamin, mineral, enzyme, trace element, micronutrient, fatty acid, triglyceride, amino acid, herbal compounds, electrolyte, protein, carbohydrate, derivative thereof or combinations thereof.

“Nutritional stores” refers to the levels of vitamins, minerals and other nutrients which will be available for use by another, developing embryo, fetus and newborn infant.

“Nutritional status” refers to the presence or absence of any nutrient deficiency, or in other words, the extent to which physiological nutrient demands are being satisfied such that deficiency is avoided.

“Optimize neurological development” refers to attainment of the highest degree of neurological development possible through natural processes without the use of any unnatural substances or procedures, such as drugs, surgery and the like.

“Specific physiological needs” refers to the unique requirements for certain levels of certain nutrients by one class of persons, such as lactating women, pregnant women, etc., as distinguished from other classes.

“Neonate” refers to the offspring of a female mammal that is nursed by said female mammal and has not yet been weaned.

“Fatty acid” refers to anyone of the paraffin series of monocarbonic acids, especially those found in animal and vegetable fats and oils, having the general formula CnH2n+1COOH. Characteristically made up of saturated or unsaturated aliphatic compounds with an even number of carbon atoms, this group of acids includes palmitic, stearic, oleic, formic and acetic acids. So called because the higher members, as stearic and palmitic acids, occur in the natural fats, and are themselves fatlike substances.

“Omega-3 fatty acid” refers to any of several polyunsaturated fatty acids found in leafy green vegetables, vegetable oils, and fish such as salmon and mackerel, capable of reducing serum cholesterol levels and having anticoagulant properties.

“Omega-6 fatty acid” refers to any of several polyunsaturated fatty acids found in vegetable oils, including walnuts and meats, capable of regulating the low density to high density lipoprotein ratio.

“Precursor” refers to a biochemical substance, such as an intermediate compound in a chain enzymatic reaction, from which a more stable or definitive product is formed; an altered form of the essential chemical before it has been transformed via biochemical reaction such as metabolism.

“Extended release” refers to the ability to continuously make available for a long or protracted period of time.

The present inventive subject matter is based, in part, upon the discovery that pregnant women have specific nutritional requirements and that there are substantial physiological benefits attained by fulfilling these requirements. Further the inventive subject matter is based upon the discovery that the ability to meet the nutritional requirements of pregnant women is sometimes hindered due to the increased sensitivity of the pregnant woman's gastrointestinal tract. However, minimizing this sensitivity is possible through implementation of lifestyle and dietary modifications. The products of the inventive subject matter provide optimum nutritional components and are provided in a dosage form which takes into account the increased gastrointestinal sensitivity of pregnant women.

Without being limited by theory, the compositions and methods of the present inventive subject matter may be effective because they are provided in a dosage form which is designed to have a low impact upon the gastrointestinal tract, in that the dosages are of soft and flexible design and minimize unpleasant taste and/or smell. Alternatively, the compositions and methods may be effective because they do not initiate, stimulate or act as catalysts to reactions having a negative effect upon the gastrointestinal tract. The present compositions are enriched with essential fatty acids. These compositions promote good health in a pregnant or lactating woman through one or more natural biological pathways. For example, the arachidonic acid cascade may play a significant role in the enrichment of the breast milk. Specifically, in the arachidonic acid cascade, linoleic acid is converted first to gamma-linolenic acid and then to further metabolites such as dihomo-gamma-linolenic acid and arachidonic acid which are precursors of 1 and 2 series prostaglandin respectively, as shown in the outline below:

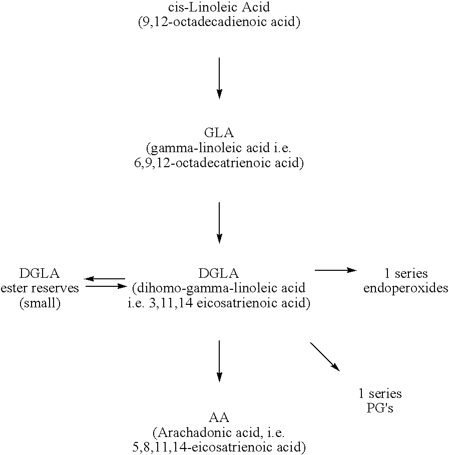

The nutritional supplements of the present inventive subject matter contain specific nutritional compositions for administration to pregnant women to alleviate nutritional deficiencies likely to occur during pregnancy. Further, the present inventive subject matter also satisfies specific vitamin and mineral requirements, the absence of which have been found to cause birth,defects, as well as provide for general health during pregnancy. The formulations of the inventive subject matter optimize the nutritional benefits of supplementation as required by the physiological stresses of pregnancy.

The nutritional compositions of the present inventive subject matter are provided in a dosage form, i.e., soft gelatin, for administration to pregnant women which minimizes unpleasant taste, regurgitation, gastroesophageal reflux, dyspepsia, nausea, or difficulty in swallowing or ingesting nutritional agents during pregnancy. The effectiveness of the soft gelatin dosage form in relation to its low impact effect upon the gastrointestinal tract appears to be related to the dosage's small size and flexible, soft physical properties. The soft gelatins of the present inventive subject matter have a smooth outer surface, which has elastic properties that provide for minimal resistance in swallowing. As such, the soft gelatins have a lesser potential to negatively impact the esophageal sphincter and thereby cause or exacerbate the condition of gastroesophageal reflux. These same properties, as well as the pre-dispersion of the nutritional compositions in the core matrix, reduce the reactivity of the actives to the acidic gastrointestinal environment, and thus lend to reduced incidences of reflux and regurgitation phenomena. Furthermore, the gelatin coating of the soft gelatins minimizes the unpleasant taste and/or smell commonly associated with traditional vitamin and mineral supplements and thereby reduces regurgitation, dyspepsia, nausea and gagging associated with these negative traits.

The nutritional compositions of the present inventive subject matter are formulated to provide for optimal health during pregnancy and to minimize any potential negative impact upon the gastrointestinal tract. The extent to which this negative impact is reduced by use of the soft gelatin formulas is mitigated by numerous external factors, such as the following non-limiting examples: stress, alcohol intake, caffeine intake, smoking, poor diet management, poor patient compliance, and the like. Moreover, the effectiveness of the compositions may vary from individual to individual for a wide array of reasons, such as genetic predisposition, health factors, and the like, without limitation.

It is difficult to quantify the minimizing effect upon unpleasant taste, regurgitation, gastroesophageal reflux, dyspepsia, nausea, or difficulty swallowing or ingesting of the soft gelatin nutritional agents. However, the average healthy pregnant woman suffering from the normal gastrointestinal disturbances associated with pregnancy, i.e., uncomplicated incidences of heartburn, gastroesophageal reflux, dyspepsia, nausea, regurgitation, gagging, and the like, without limitation, may be able to minimize these symptoms through use of the present formulations. Furthermore, even for pregnant women who are experiencing gastrointestinal disturbances to a more pronounced than what would be classified as “normal” may find the formulations of the present inventive subject matter have a positive effect upon these symptoms, particularly where the gastrointestinal distress is caused or exacerbated by the ingestion of traditional vitamin and mineral tablets or where their condition has made it impossible to ingest traditional tablet form prenatal supplements.

The present inventive subject matter contemplates the inclusion of a viscous biologically-active core composition which is comprised of a nutritional compound uniformly suspended in an edible oil or a polymer. Preferably, the nutritional compound is about 2 percent to 98 percent by weight of the biologically-active core composition. More preferably, the nutritional compound is about 3 percent to 97 percent by weight of the biologically-active core. Most preferably, however, the nutritional compound is about 4 percent to 96 percent by weight of the biologically-active core.

The formulations of the present inventive subject matter contain vitamin B₆ or derivatives thereof. Derivatives of vitamin B₆ include compounds formed from vitamin B₆ which are structurally distinct from vitamin B₆, but which retain the active function of vitamin B₆. Such derivatives include, without limitation, pyridoxine, pyridoxine hydrochloride, salts of vitamin B₆, alkaline salts of vitamin B₆, chelates of vitamin B₆, combinations thereof and the like. The vitamin B₆ may be present in a single form or in various different forms in combination within the present compositions. The specific amount of vitamin B₆ in the compositions is adjusted based on the type of dosage form utilized, i.e., immediate release or controlled release.

In the case of the immediate release compositions, the amounts of vitamin B₆ in the compositions preferably range from about 1 mg to about 115 mg. More preferably, the amounts of vitamin B₆ in the immediate release compositions range from about 2 mg to about 110 mg. Even more preferably, the amounts of vitamin B₆ in the immediate release compositions range from about 3 mg to about 107 mg. Most preferably, the amounts of vitamin B₆ in the immediate release compositions range from about 4 mg to about 105 mg.

The amount of vitamin B₆ present in the controlled release compositions of the present-inventive subject matter, preferably range from about 75 mg to about 125 mg. More-preferably; the amount of vitamin B₆ in the controlled release compositions is about 85 mg to about 115 mg. Even more preferably, the amount of vitamin B₆ in the controlled release compositions is about 90 mg to about 110 mg. Most preferably, the amount of vitamin B₆ in the controlled release compositions is about 95 mg to about 105 mg.

The compositions of the present inventive subject matter may include a folic acid compound or derivative thereof. The derivatives of folic acid include folacin, pteroylglutamic acid, as well as compounds formed from folic acid which are structurally distinct from folic acid, but which retain the active function of folic acid. Non-limiting examples of such derivatives include: salts of folic acid, chelates of folic acid, combinations thereof and the like. The folic acid may be present in a single form or in various different forms in combination within the present compositions. Folic acid in the present compositions may be presented in various types of dosage forms, for example and without limitation, immediate release or controlled release. Extended release folic acid may be included in the present compositions, because such folic acid minimizes gastrointestinal side effects. The amounts of folic acid preferably range from about 0.1 mg to about 8. More preferably, the amount of folic acid in these compositions is about 0.2 mg to about 5 mg.

The compositions of the present inventive subject matter may include a calcium compound or derivative thereof. The addition of calcium is beneficial nutritionally, and the calcium compound minimizes stomach upset, as well as increases the bioavailability of folic acid when present in the composition. The derivatives of calcium include, without limitation, calcium carbonate, calcium sulfate, calcium oxide, calcium hydroxide, calcium apatite, calcium citrate-malate, calcium gluconate, calcium lactate, calcium phosphate, calcium threonate, calcium levulinate, bone meal, oyster shell, as well as compounds formed from calcium which are structurally distinct from calcium, but which retain the active function of calcium. Non-limiting examples of such derivatives include: salts of calcium, chelates of calcium, combinations thereof and the like. The calcium may be present in a single form or in various different forms in combination within the present compositions. Calcium is preferably present in the composition of the present inventive subject matter in an amount ranging from about 1 mg to about 2,500 mg and maybe in an immediate or controlled release form. More preferably, calcium is present in an amount ranging from about 10 mg to about 2,000 mg.

The compositions of the present inventive subject matter include omega-3 and omega-6 fatty acids and precursors to omega-3 and omega-6 fatty acids from any source, including, without limitation, natural or synthetic oils, fats, waxes or combinations thereof. Moreover, the fatty acids herein may be derived, without limitation, form nonhydrogenated oils, partially hydrogenated oils, fully hydrogenated oils, or combinations thereof. Nonlimiting exemplary sources of fatty acids include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba oil, tallow, beef tallow, butter, chicken fat, lard, dairy butter fat, shea butter, or combinations thereof. Specific non-limiting exemplary fish or marine oils include shell fish oil, tuna oil, mackerel oil, salmon oil, menhaden oil, anchovy oil, herring oil, trout oil, sardine oil, or combinations thereof.

Preferably, the precursor of omega-3 fatty acid and the precursor of omega-6 fatty acid are linolenic acid and linoleic acid, respectively. Also preferable are precursors of omega-3 and omega-6 fatty acids which are derived from a plant source including, without limitation sunflower oil, soybean oil, flaxseed oil linseed oil and vegetable shortening. In-addition, most preferable are omega-6 fatty acids, i.e., decosahexaenoic acid (DHA), derived from marine sources including, without limitation, from fish oils such as tuna, mackerel, menhaden, anchovy, herring, trout sardine and salmon, as well as shellfish oil.

Omega-3 and omega-6 fatty acid precursors are biochemical substances which precede and are forerunners to the more stable and definitive products, i.e., omega-3 and omega-6 fatty acids. These biochemical 'substances, include, without limitation, linolenic acid, eicosapentaenoic acid, and linoleic acids, DHA.

The presence of fatty acids in the supplements is significant for various reasons. First, the body derives most of its energy from triglycerides, a molecule of glycerol with three fatty acids attached. The stored fatty acids support most of life's activities when individual's are pregnant, between meals or must go without food. While the body can make many fatty acids, it cannot make linoleic acid or linolenic acid. These two fatty acids are indispensable to body functions and therefore must be supplied through the diet.

Secondly, essential fatty acids are important for pregnant or lactating women for development of the brain, immunological system and cardiovascular system, and have some role to play in every organ of the body of the fetus or nursing infant. Linoleic acid is the most important member of the omega-6 family of fatty acids. The body uses linoleic acid to synthesize an important 20-carbon fatty acid, arachidonic acid, which helps maintain the structural integrity of cell membranes. Combinations of both plant and marine derived sources of omega fatty acids are advantageous and valuable because plant sources have only omega-3 and omega-6 precursors for fatty acids (linolenic and linoleic acids) whereas marine oils contain the preformed omega-3 and omega-6 acids themselves, eicosapentaenoic acids (EPA) and decosahexaenoic acids (DHA). While the use of precursors of these essential fatty acids is valuable from a biological sense, biological energy and time must be expended in order to biotransform these precursors into the essential fatty acids. By using a combination of both plant and marine oils the presence of the essential fatty acids themselves allows for immediate utilization, whereas the plant precursors allows for the later biotransformation and utilization of the essential fatty acids.

The present inventive subject matter is based, in part, on the discovery that when compositions having certain fatty acids, in certain amounts and proportions to one another, are administered to pregnant or lactating women help to ensure that the mother has adequate essential fatty acids for her own use and for the developing fetus. The fatty acid supplement may also further contain vitamins and minerals to confer added health benefits to the fetus and mother.

The two fatty acid compounds are present in the composition in critical proportions to one another. Preferably, the weight ratio of the precursor of omega-3 fatty acid and the precursor of omega-6 fatty acid to the omega-6 fatty acid is about 1:4. More preferably, the weight ratio of the precursor of omega-3 fatty acid and the precursor of omega-6 fatty acid to the omega-6 fatty acid is about 1:2.5 to 3.0.

The fatty acids of the present inventive subject matter may be used as such or as biologically acceptable and physiologically equivalent derivatives as, for example, detailed later herein. Reference to any of the fatty acids including reference in the claims is to be taken as including reference to the acids when in the form of such derivatives. Equivalence is demonstrated by entry into the biosynthetic pathways of the body as evidenced by effects corresponding to those of the acids themselves or their natural glyceride esters. Thus, indirect identification of useful derivatives is by their having the valuable effect in the body of the fatty acid itself, but conversion, for example, of gamma-linolenic acid to dihomo-gamma-linolenic acid and on to arachidonic acid can be shown directly by gas chromatographic analysis of concentrations in blood, body fat, or other tissue by standard techniques, well known to persons of ordinary skill in the art to which the present inventive subject matter pertains.

Derivatives of linoleic acid, as used in the present inventive subject matter, include, without limitation, salts of linoleic acid, alkaline salts of linoleic acid, esters of linoleic acid, and combinations thereof Derivatives of linolenic acid, as used in the present inventive subject matter, include, without limitation, salts of linolenic acid, alkaline salts of linolenic acid, esters of linoleic acid, and combinations thereof. The salts and alkaline salts here in refer to those regularly used organic or inorganic salts which are acceptable for pharmaceutical use. Non-limiting exemplary linolenic acids include gamma-linoleic acid and dihomo-gamma-linolenic acid.

Linolenic acid is the most important member of the omega-3 family of fatty acids. The body requires this fatty acid to make eicosapentaenoic acid (EPA) and docosahexaenoic acid (DHA). Many body tissues require EPA and DHA. DHA is especially important in the retina and in the cerebral cortex of the brain. Half of the DHA in a fetuses body accumulates in the brain before birth, and half after birth, an indication of the importance of fatty acids to the fetus during pregnancy and then to the young infant during lactation.

The fatty acids in the present compositions are derived from both plant and animal sources. Combinations of both plant and marine sources of fatty acids are beneficial, because plant derived sources contain only the omega-3 and omega-6 precursors to linolenic and linoleic acids, while marine sources contain linolenic and linoleic acids. Thus, while the body transforms the plant derived precursors for use, it utilizes the immediately available marine sources of linolenic and linoleic acids.

The compositions of the present inventive subject matter may include a vitamin E compound or derivative thereof. The derivatives of vitamin E include, without limitation, alpha-tocopherol, DL alpha tocopherol, DL alpha tocopheryl acetate, tocopherol, tocotrienol, as well as compounds formed from vitamin E which are structurally distinct from vitamin E, but which retain the active function of vitamin E. Non-limiting examples of such derivatives include: salts of vitamin E, alkaline salts of vitamin E, chelates of vitamin E, combinations thereof and the like. The vitamin E may be present in a single form or in various different forms in combination within the present compositions. Preferably, the compositions of the present invention contain about 10 IU to about 800 10 of vitamin E. More preferably, the compositions of the present invention contain about 25 IU to about 600 IU of vitamin E.

The compositions of the present inventive subject matter may optionally include one or more of the following vitamins or derivatives thereof, without limitation: biotin, vitamin B₁, thiamin, thiamin pyrophosphate, vitamin B₂, riboflavin, flavin mononucleoride, flavin adenine dinucleotide, vitamin B₃, niacin, nicotinic acid, nicotinamide, niacinamide, nicotinamide adenine dinucleotide, tryptophan, biotin, pantothenic acid, vitamin B₁₂, cobalamin, methylcobalamin, deoxyadenosylcobalamin, cyanocobalamin, vitamin C, ascorbic acid, vitamin A, retinol, retinal, retinoic acid, beta-carotene, vitamin D, vitamin D₃, calciferol, cholecalciferol, dihydroxy vitamin D, 1,25-dihydroxycholecalciferol, 7-dehyrdocholesterol, vitamin K, menadione, menaquinone, phylloquinone, and naphthoquinone.

The compositions of the present inventive subject matter may optionally include one or more of the following minerals and/or trace minerals or derivatives thereof, without limitation: phosphorus, potassium, sulfur, sodium, docusate sodium, chloride, magnesium, magnesium stearate, magnesium carbonate, magnesium oxide, magnesium hydroxide, magnesium sulfate, manganese, copper, cupric sulfate, iodide, boron, zinc, zinc oxide, chromium, molybdenum, iron, carbonyl iron, ferric iron, ferrous fumarate, polysaccharide iron, fluoride, selenium, molybdenum, cobalt and combinations thereof and derivatives thereof, without limitation. Non-limiting exemplary derivatives of mineral compounds include salts, alkaline salts, esters and chelates of any mineral compound.

The compositions of the present inventive subject matter may optionally include one or more of the following drug categories, in nonteratogenic formulation, without limitation: analgesics, such as acetaminophen, antacids, calcium antacids, magnesium antacids, antibiotics, antihistamines, salicylates, hormonal agents and the like.

The present inventive subject matter may include an edible oil such as one of the following non-limiting examples: seed oil, nut oil, fish oil, vegetable oil, safflower oil, sunflower oil, olive oil, soybean oil, corn oil, safflower oil, olive oil, soybean oil, corn oil, peanut oil, cotton seed oil, palm oil, cocoa oil, coconut oil, flax seed oil, palm kernel oil, canola oil, grape seed oil, walnut oil, sesame oil, cod liver oil, tuna oil, salmon oil, mackerel oil and combinations thereof and derivatives thereof.

The present inventive subject matter may include a polymer, such as one of the following non-limiting examples: polyethylene glycol, propylene glycol, glycerin, polyvinylpyrrolidone, lecithin, PEO, polymeric cellulose esters, copolymeric cellulose esters, cellulose derivatives, acrylate, hydrogenated vegetable oils, natural and synthetic waxes and combinations thereof.

The present inventive subject matter may further include a surfactant such as sodium lauryl sulfate, synthetic ionic surfactant, a synthetic nonionic surfactant, a nonsynthetic ionic surfactant, a nonsynthetic nonionic surfactant, polysorbate 80, polysulfated glucosoglycans, glucosaminoglycans, mucopolysaccharides, derivatives and mixtures thereof and the like, without limitation.

It is also possible in the nutritional composition of the present inventive subject matter for the dosage form to combine various forms of release, which include, without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan. Each of these specific techniques or procedures does not constitute an inventive aspect of this inventive subject matter.

The methods of the present inventive subject matter contemplate dosage forms involving the administration of a nutritional composition in a single dose during a 24 hour period of time, a double dose during a 24 hour period of time, or more than a double dose during a 24 hour period of time. The dosing may be taken simultaneously or at different times depending on the prescribed dosage.

The present inventive subject matter contemplates the use of pharmaceutically acceptable carriers which may be prepared from a wide range of materials. Without being limited thereto, such materials include diluents, binders and adhesives, lubricants, plasticizers, disintegrants, colorants, bulking substances, flavorings, sweeteners, fragrances, aromatics, edible oils, polymers and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives such as whey, starches, and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluable hydrophobic substances, such as diethyl phthalate, diethyl sebacate and caster oil are used to delay the release of water-soluble vitamins, such as vitamin B₆ and vitamin C. In contrast, hydrophilic plasticizers are used when water-insoluble vitamins are employed which aid in dissolving the encapsulated film, making channels in the surface, which aid in nutritional composition release.

Flavorings utilized in the nutritional supplements of the present inventive subject matter can be in the form of flavored extracts, volatile oils, and any other commercially available flavoring, without limitation. Nonlimiting examples of flavorings include: pure anise extract, pure vanilla extract, pure lemon extract, pure orange extract, pure peppermint extract, pure spearmint extract, pure ginger extract, imitation banana extract, imitation cherry extract, imitation strawberry extract, imitation raspberry extract, imitation pineapple extract, imitation peach extract, imitation apple extract, imitation coconut extract, vanillin, imitation guava extract, imitation mango extract, balm oil, bay oil, bergamot oil, cinnamon oil, cherry oil, clove oil, peppermint oil, spearmint oil, cedarwood oil, cocoa oil derivatives thereof and combinations thereof.

The compositions of the present inventive subject matter contemplate formulations of various viscosities. The viscous stresses in liquids arise from intermolecular reaction. The concept of viscosity in relation to soft gelatin medicament formulations is important when it is considered that viscosity is used as an index of the suitability of a particular formulation for a particular purpose, i.e., the suitability of a biologically-active core for insertion into a soft gelatin shell.

The centipoise unit is frequently used to measure the dynamic viscosity of mobile liquids and is the unit basis contemplated by the present inventive subject matter. The formal definition of viscosity is derived from a Newtonian theory, wherein under conditions of parallel flow, the shearing stress is proportional to the velocity gradient. If the force acting on each of the two planes of area A parallel each other, moving parallel to each other with a relative velocity V, and separated by a perpendicular distance X, be denoted by F, the shearing stress is F/A and the velocity gradient, which will be linear for a true liquid, is V/X. Thus, F/A=n V/X, where the constant n is the viscosity coefficient or dynamic viscosity of the liquid. Van Nostrand's Scientific Encyclopedia, 2891 (6th Ed. 1983).

Formulations falling within the scope of the present inventive subject matter may be prepared by methods well known to those of skill in the art, without limitation. For example, without limitation, formulations falling within the scope of the present inventive subject matter may be prepared by dispersing the active substance in an appropriate vehicle, such as vegetable oil or the like, to form a high viscosity mixture. Preferably, the viscosity of the mixture would range from about 1,000 centipoise to about 1.5 million centipoise. Even more preferably, the viscosity of the mixture would range from about 20,000 centipoise to about 130,000 centipoise. Preferably, the viscosity of the mixture would range from about 20,000 centipoise to about 60,000 centipoise. This mixture is then encapsulated with a gelatin based film using technology and machinery known to persons of ordinary skill in the art. The industrial units so formed are then dried to a constant weight and stored for future use.

The forgoing is considered as illustrative only of the principles of the inventive subject matter. Further, since numerous modification and changes will readily occur to those skilled in the art, it is not desired to limit the inventive subject matter to the exact construction and operation shown and described, and accordingly all suitable modifications and equivalents may be restored to, falling within the scope of the-inventive subject matter.

The following examples are illustrative of preferred embodiments of the inventive subject matter and are not to be construed as limiting the inventive subject matter thereto. All percentage are based on the percent by weight of the final delivery system or formulation prepared unless otherwise indicated and all totals equal 100% by weight.

### EXAMPLES

Preparation of Soft Gel Nutritional Supplement

### Example 1

The following compositions were used to prepare soft gelatin prenatal supplements:

TABLE I

| | FORMUL A |
|---|---|
| ACTIVE | AMOUNT |
| Folic Acid, mg | 1.8 |
| Vitamin B₆, mg | 75.0 |
| Vitamin B₁₂, mg | 12.0 |
| Soybean Oil, mg | 4.95 |
| Mineral Oil, mg | 50.0 |
| Fish Oil, mg | 181.5 |
| Linolenic Acid, mg | 35.0 |
| Linoleic Acid, mg | 35.0 |
| Vitamin E, mg | 418.2 |
| Propylene Glycol, mg | 20.0 |
| Polysorbate 80, mg | 3.0 |
| Silicon, mg | 50.0 |
| Wax,* mg | 100.0 |
| Polyethylene Oxide, mg | 50.0 |

*The wax component may be either natural or synthetic.

Soft gelatins incorporating the above formulations were prepared using conventional methods and materials known in the pharmaceutical art. The resulting soft gelatins were recovered and stored for future use.

### Example 2

The following compositions were used to prepare soft gelatin prenatal supplements:

TABLE II

| ACTIVE | FORMUL A AMOUNT |
|---|---|
| Folic Acid, mg | 1.0 |
| Vitamin B₆, mg | 50.0 |
| Vitamin B₁₂, mg | 12.0 |
| Niacin, mg | 20.0 |
| Riboflavin, mg | 3.4 |
| Thiamin, mg | 3.0 |
| Iron, mg | 45.0 |
| Zinc oxide, mg | 15.0 |
| Copper, mg | 2.0 |
| Magnesium, mg | 100.0 |
| Soybean Oil, mg | 4.95 |
| Mineral Oil, mg | 50.0 |
| Fish Oils, mg | 181.5 |
| Linolenic Acid, mg | 35 |
| Linoleic Acid, mg | 35 |
| Vitamin E, mg | 39.8 |
| Calcium, mg | 275 |
| Vitamin D₃, mg | 0.19 |
| Propylene Glycol, mg | 20 |
| Polysorbate 80, mg | 3 |
| Silicon, mg | 50 |
| Wax,* mg | 100 |
| Polyethylene Oxide, mg | 50 |

*The wax component may be either natural or synthetic.

Soft gelatins incorporating the above formulations were prepared using conventional methods and materials known in the pharmaceutical art. The resulting soft gelatins were recovered and stored for future use.

### Example 3

A soft gelatin supplement is prepared, by first combining mineral oil and soybean oil in a first vessel and blending it to form a uniform oil mixture, heating the oil mixture to 45 degrees Celsius, and then adding propylene glycol. In a second vessel preheated to 70 degrees Celsius, yellow beeswax and soybean oil are added and blended until a uniform wax mixture is formed. The wax mixture is cooled to 35 degrees Celsius and then added to the oil mixture. To this combined oil and wax mixture, folic acid, vitamin B₆, iron, magnesium, and calcium are then added and blended together to form a uniform biologically active mixture. This mixture is then cooled to 30 degrees Celsius to form a viscous biologically active core composition, after which time the composition is ready for encapsulation in a soft gelatin shell.

A soft gelatin shell is prepared by heating purified water in a suitable vessel and then adding gelatin. This water gelatin mixture is mixed until the gelatin is fully dissolved, and then glycerin, preservatives, one or more flavors, and one or more colorants are added. This gelatin mixture is blended well and cooled. The shells are then filled with the core composition and formed in accordance with soft gelatin techniques commonly used and well known to persons of skill in the art.

The invention being thus described, it will be apparent that the same may be varied in many ways. Such variations are not to be regarded as a departure from the spirit and scope of the invention, and all such modifications are intended to be within the scope of the appended claims.

We claim:

1. A soft gelatin nutritional supplement for administration to a pregnant or lactating woman for the purpose of minimizing unpleasant taste, regurgitation, gastroesophageal reflux, dyspepsia, nausea, or difficulty in swallowing or ingesting nutritional agents, which comprises:
an omega-3 fatty acid precursor, a derivative thereof, or mixtures thereof;
an omega-6 fatty acid, a derivative thereof, or mixtures thereof;
an omega-6 fatty acid precursor, a derivative thereof, or mixtures thereof;
wherein the weight ratio of said omega-3 fatty acid precursor and said omega-6 fatty acid precursor to said omega-6 fatty acid is about 1:2.5 to 3.0; and
a soft gelatin shell.

2. The supplement of claim 1, wherein said omega-3 fatty acid precursor and said omega-6 fatty acid precursor are derived from a plant source selected from the group consisting of flaxseed oil, linseed oil, soybean oil, sunflower oil and combinations thereof.

3. The supplement of claim 1, wherein said omega-3 fatty acid precursor is linolenic acid.

4. The supplement of claim 1, wherein said omega-6 fatty precursor is linoleic acid.

5. The supplement of claim 1, wherein said omega-6 fatty acid is derived from a marine source selected from the group consisting of shellfish oil, tuna oil, mackerel oil, salmon oil, menhaden, anchovy, herring, trout, sardines and combinations thereof.

6. The supplement of claim 1, wherein said omega-6 fatty acid is docosahexaenoic acid.

7. The supplement of claim 1, wherein said nutritional supplement is additionally comprised of a folic acid compound or derivative thereof in amounts ranging from about 0.2 mg to about 5.0 mg.

8. The supplement of claim 7, wherein said folic acid compound is extended release.

9. The supplement of claim 1, wherein said nutritional supplement is additionally comprised of a vitamin E compound, a derivative thereof or mixtures thereof in amounts from about 200 IU to about 600 IU.

10. The supplement of claim 1, wherein said nutritional supplement is additionally comprised of a mineral compound selected from the group consisting of iron, calcium, magnesium, potassium, copper, chromium, zinc, molybdenum, iodine, boron, selenium, manganese, derivatives thereof and combinations thereof.

11. The supplement of claim 1, wherein said nutritional supplement may be additionally comprised of one or more vitamin compounds selected from the group consisting of vitamin A, thiamine, niacinamide, pyridoxine, riboflavin, cyanocobalamin, biotin, pantothenic acid, vitamin C, vitamin D, vitamin E, vitamin K, derivatives thereof and combinations thereof.

12. A soft gelatin nutritional supplement for administration to a pregnant or lactating woman for the purpose of minimizing unpleasant taste, regurgitation, gastroesophageal reflux, dyspepsia, nausea, or difficulty in swallowing or ingesting nutritional agents, which comprises:
an omega-3 fatty acid precursor, a derivative thereof, or mixtures thereof;
an omega-6 fatty acid, a derivative thereof, or mixtures thereof;
an omega-6 fatty acid precursor, a derivative thereof, or mixtures thereof;
a calcium compound, a derivative thereof, or mixtures thereof in an amount ranging from about 10 mg to about 2,000 mg;
wherein the weight ratio of said omega-3 fatty acid precursor and said omega-6 fatty acid precursor to said omega-6 fatty acid is about 1:2.5 to 3.0; and
a soft gelatin shell.

13. The supplement of claim 12, wherein said omega-3 fatty acid precursor and said omega-6 fatty acid precursor are derived from a plant source selected from the group consisting of flaxseed oil, linseed oil, soybean oil, sunflower oil and combinations thereof.

14. The supplement of claim 12, wherein said omega-3 fatty acid precursor is linolenic acid.

15. The supplement of claim 12, wherein said omega-6 fatty acid precursor is linoleic acid.

16. The supplement of claim 12, wherein said omega-6 fatty acid is derived from a marine source selected from the group consisting of shellfish oil, tuna oil, mackerel oil, salmon oil, menhaden, anchovy, herring, trout, sardines and combinations thereof.

17. The supplement of claim 12, wherein said omega-6 fatty acid is docosahexaenoic acid.

18. The supplement of claim 12, wherein said nutritional supplement is additionally comprised of a folic acid compound or derivative thereof in amounts ranging from about 0.2 mg to about 5.0.mg.

19. The supplement of claim 18, wherein said folic acid is extended release.

20. The supplement of claim 12, wherein said nutritional supplement is additionally comprised of a vitamin E compound, a derivative thereof or mixtures thereof in amounts from about
200 IU to about 600 IU.

21. The supplement of claim 12, wherein said nutritional supplement is additionally comprised of a mineral compound selected from the group consisting of iron, magnesium, potassium, copper, chromium, zinc, molybdenum, iodine, boron, selenium, manganese, derivatives thereof and combinations thereof.

22. The supplement of claim 12, wherein said nutritional supplement may be additionally comprised of one or more vitamin compounds selected from the group consisting of vitamin A, thiamine, niacinamide, pyridoxine, riboflavin, cyanocobalamin, biotin, pantothenic acid, vitamin C, vitamin D, vitamin E, vitamin K, derivatives thereof and combinations thereof.

23. A method for reducing unpleasant taste, regurgitation, gastroesophageal reflux, dyspepsia, or nausea associated with the administration of nutritional supplements, which comprises:
orally administering to a pregnant or lactating woman a soft gelatin capsule, wherein said soft gelatin capsule comprises:
an omega-3 fatty acid precursor, a derivative thereof, or mixtures thereof;
an omega-6 fatty acid, a derivative thereof, or mixtures thereof;
an omega-6 fatty acid precursor, a derivative thereof, or mixtures thereof;
wherein the weight ratio of said omega-3 fatty acid precursor and said omega-6 fatty acid precursor to said omega-6 fatty acid is about 1:2.5 to 3.0.
